(19) United States
(12) Patent Application Publication
(10) Pub. No.: US 2009/0081808 A1
(43) Pub. Date: Mar. 26, 2009

(54) DEVICE AND METHOD FOR IDENTIFYING MYCOTOXINS

(75) Inventors: Jens Burmeister, Leverkusen (DE); Ingmar Dorn, Koln (DE); Uwe Rabe, Leverkusen (DE); Isolde Hauser-Hahn, Leverkusen (DE)

Correspondence Address: NORRIS, MCLAUGHLIN & MARCUS, PA, 875 THIRD AVENUE, 18TH FLOOR, NEW YORK, NY, 10022, US

(73) Assignees: BAYER TECHNOLOGY SERVCES GMBH, Leverkusen (DE); BAYER CROPSCIENCE AG, Monheim (DE)

(21) Appl. No.: 12/158,788

(22) PCT Filed: Dec. 13, 2006
(86) PCT No.: PCT/EP2006/012000

§ 371 Date: Nov. 17, 2008

(30) Foreign Application Priority Data
Dec. 23, 2005 (DE) 10 2005 062 377.8

(51) Int. Cl. G01N 33/566; G01N 21/00

(52) U.S. Cl. 436/501; 422/82.11

(57) ABSTRACT

The invention relates to an apparatus and a process for detection of mycotoxins and to kits suitable for carrying out said process.

The invention relates to an apparatus and a process for detection of mycotoxins and to kits suitable for carrying out said process.

Detection of mycotoxins comprises a large field of application, for example in the food and feed sectors, in environmental analysis, in crop protection and in biochemical research.

Mycotoxins are toxins produced by molds, which have very different chemical structures. Mycotoxins are found in harvest products such as grain, oil-containing seeds and fruits, and may cause poisoning of humans and animals. Over 300 different mycotoxins have been identified by now which are classified into approx. 25 structural types and exhibit different toxic actions. Depending on the type of toxin, mycotoxins can bring about acute or chronic poisoning. Common groups of mycotoxins are aflatoxins, ochratoxins, ergot alkaloids, patulin and fusarium toxins. Particularly important among the fusarium toxins are deoxynivalenol, zearalenone, nivalenol, T-2-/HT2 toxin and the fumonisins because they are frequently found in cereal products. Accordingly, an assay for mycotoxins, for example for toxins of field fungi, for example fusarium toxins, or for toxins of storage fungi, should be carried out in granaries, grain-trading and grain-processing businesses, for example mills, malt houses, feed-producing businesses, agricultural businesses, advice centers, universities or government departments, for example the department for consumer protection, in order to ensure food quality.

A number of processes for detecting mycotoxins have been disclosed in the prior art. Mycotoxins are detected, for example, by chromatographic processes such as HPLC, which may be coupled with fluorescence-based, absorptive or mass-spectrometric detection. Prior to HPLC analysis, for example of a grain sample, the analyte is usually concentrated and purified by means of immunoaffinity columns. All HPLC-based processes have the disadvantages of great capital expenditure, relatively complex sample handlng and prolonged analyses. Owing to said disadvantages, HPLC-based detection processes are not suitable for rapid, inexpensive and simple analysis, for example of grain samples in businesses producing, accepting, trading or processing grain. HPLC-based analysis is carried out instead in specialized, analytical laboratories. Consequently, in practice, the result is available only after a delay of several days.

An alternative method of detecting mycotoxins is the ELISA (enzyme linked immunosorbent assay) technology. The ELISA is provided with microtiter plates whose wells are coated, for example, with capture antibodies which specifically bind to a mycotoxin. Disadvantages of the ELISA are the many pipetting, washing and incubation steps which may result in relatively long analyses of more than 30 minutes. This prevents the assay being carried out rapidly on the spot outside an analytical laboratory. Moreover, the assay does not allow simultaneous detection of multiple analytes, since each microtiter plate is usually coated only with one type of antibodies.

Another method of detecting mycotoxins are lateral flow assays (LFAs). Mycotoxins may be detected by means of LFA, for example, by performing a direct, competitive immunoassay on a nitrocellulose strip, with the sample to be analyzed being pulled through the entire nitrocellulose strip due to capillary forces. Disadvantageously, the process permits only qualitative mycotoxin detection. Another disadvantage of this assay is the requirement of a separate strip for each mycotoxin.

The prior art also includes studies on the development of processes for detecting mycotoxins, for example described by M. M. Ngundi et al., Anal. Chem. 2005, 77, 148-154. This process comprises carrying out an indirect, competitive immunoassay for detecting ochratoxin A by immobilizing ochratoxin A on glass slides. The mixture of a fluorescently labeled antibody to ochratoxin A and of the sample to be determined is applied to the slide which can be read out after the unbound antibodies have been removed by washing. Disadvantageously, this process requires washing steps and incubation times of from 10 to 20 minutes and also complicated fluorescence imaging systems for reading out the results. As a result, it is not possible to develop a rapid assay on this basis that can be carried out on the spot outside an analytical laboratory.

The processes known in the prior art for detecting mycotoxins thus require great capital expenditure owing to complicated read-out apparatus, include many manual steps or cannot be used outside an analytical laboratory.

The object of the present invention is therefore to provide a process which overcomes at least one of the abovementioned disadvantages of the prior art, in particular a process which enables mycotoxins to be detected in a sample in a rapid, inexpensive and easy to carry out manner.

This object is achieved by a process for rapid detection of mycotoxins, comprising the following steps:
a) providing a thin-film waveguide comprising a first optically transparent wave-guiding layer (a) on top of a second optically transparent layer (b), with (b) having a lower refractive index than (a), to which waveguide specific and/or affinity binding partners are immobilized as a chemical or biochemical recognition element for mycotoxins and/or a binding partner in a spatially separated manner,
b) applying a mycotoxin(s)-containing sample and binding partners to the immobilized binding partners on said thin-film waveguide,
c) detecting a signal in the evanescent field due to the interaction of the binding partners immobilized on the thin-film waveguide with the mycotoxins from the sample and/or with the binding partners,
d) determining the amount of mycotoxin(s) present in the sample.

The present invention further relates to an apparatus for carrying out the process for detection of mycotoxins.

A further subject matter is a kit suitable for carrying out the process for detection of mycotoxins.

Further advantageous embodiments of the invention arise from the dependent claims.

Surprisingly, it was found that the process of the invention for detection of mycotoxins can be carried out readily and outside specialized analytical laboratories. This enables the process of the invention to be carried out by way of a rapid assay, without necessarily handing over samples to a laboratory for analysis. Furthermore, mycotoxin detection according to the process of the invention advantageously requires only a few, if any, washing steps. This is particularly advantageous in that carrying out washing steps is time-consuming, prolongs the time until a result of the analysis is obtained, and may distort the results of the analysis or even render detection wholly impossible, in particular when the latter is carried out with little care or improperly.

Combining the advantageous properties of the process of the invention enables mycotoxins to be detected in food items, for example in granaries, in grain-trading or grain-processing businesses. In particular, the process can be carried out easily and rapidly and this enables even individuals who are not specialized analysts of a specialist laboratory to carry out said process.

Preferred embodiments of the process make use of a thin-film waveguide in the form of an evanescent field biochip based on a thin-film waveguide, preferably a planar optical waveguide biochip based on a thin-film waveguide.

Optical waveguides are a class of signal transducers which can be used for detecting the change in the optical properties of a medium bordering a wave-guiding layer, typically a dielectric. When light is transported in guided mode within the wave-guiding layer, the light field does not decrease abruptly at the medium/waveguide interface but rather decays exponentionally in the detection medium adjacent to the waveguide. This exponentionally decaying light field is referred to as evanescent field. A change in the optical properties of the medium bordering the waveguide within the evanescent field can be detected using a suitable measurement setup.

The use of waveguides as signal transducers is advantageous in that, in the case of recognition elements immobilized at the waveguide interface, binding to or the reaction of said recognition element can be detected when the optical properties of the detection medium change at the interface with the waveguide.

Accordingly, it is possible both to save time and to simplify the procedure when carrying out said detection.

Thus a signal or a labeled element can be detected by way of the changing optical properties of the medium, for example of a sample to be analyzed, directly on the surface of the signal transducer or thin-film waveguide, for example by way of a change in absorbence, fluorescence, phosphorescence, luminescence or the like.

Preference is given to detecting a fluorescence signal in the evanescent field. Labeling elements which may be used according to the invention for labeling the binding partners, for example mycotoxins, mycotoxin conjugates, antibody conjugates or antibodies, are preferably organic fluorophores, nanoparticles, fluorescent nanoparticles, beads, fluorescent beads, fluorescent proteins or other signaling molecules or units or any combinations of various labeling elements. Preference is given to using binding partners which have been labeled in a luminescence-capable manner. Preferred labeling elements are organic fluorophores and/or fluorescent proteins.

According to the process of the invention, the preferably fluorescent labeled binding partner may be excited by an evanescent field. In preferred embodiments, the evanescent field is generated by a planar optical waveguide as described in U.S. Pat. No. 5,959,292, Duveneck et al. Isotropically emitted fluorescence can be detected using a suitable setup. In other embodiments, fluorescence coupled into the waveguide may be coupled out of the waveguide again by a suitable optical element and be detected using a suitable optical setup.

Particularly advantageously, washing off preferably fluorescently labeled binding partners or a sample or solution containing labeled binding partners prior to detection of a signal may be restricted or even entirely be dispensed with. This enables mycotoxins to be detected in less time as well as in a simplified manner, since providing the various buffer solutions of the washing protocol which are normally used can also be dispensed with.

Usable thin-film waveguides preferably comprise an optically transparent wave-guiding layer (a) comprising oxides selected from the group comprising TiO₂, ZnO, Nb₂O₅, Ta₂O₅, HfO₂ and/or ZrO₂, preferably selected from the group comprising TiO₂, Ta₂O₅ and/or Nb₂O₅. Preferably, the optically transparent wave-guiding layer (a) is made of TiO₂, ZnO, Nb₂O₅, Ta₂O₅, HfO₂ or ZrO₂, preferably TiO₂, Ta₂O₅ or Nb₂O₅. The use of tantalum pentoxide has proved particularly advantageous, in particular for detection of a fluorescence signal.

Particular embodiments comprise applying to the thin-film waveguide, in particular to the optically transparent wave-guiding layer (a) comprising oxides selected from the group comprising TiO₂, ZnO, Nb₂O₅, Ta₂O₅, HfO₂ and/or ZrO₂, mono- or multilayers of organophosphoric acids of the following formula (I)

R—OPO₃H₂ (I)

and/or organophosphonic acids of the following formula (II)

R—PO₃H₂ (II)

and/or their salts, where

R is a C₁₀ to C₂₄ alkyl.

Preferably usable are organophosphoric acids and/or organophosphonic acids, preferably organophosphates and/or organophosphonates, with R being selected from the group comprising unbranched C₁₀ to C₂₀ alkyl, preferably selected from the group comprising unbranched C₁₂ to C₁₈ alkyl, preferably selected from the group comprising dodecylphsophoric acid, dodecylphosphate, octadecylphosphonate and/or octadecylphosphonic acid.

Preferably usable are organophosphoric acids or organophosphates which may be applied to the thin-film waveguide by way of water-soluble salts from an aqueous solution.

In preferred embodiments, the organophosphoric acids and/or organophosphonic acids, preferably organophosphates, are applied by way of a monolayer to the thin-film waveguide, in particular an evanescent field biochip, preferably a planar optical waveguide biochip. They may be applied by means of dipping processes.

The monolayer may be applied as an adhesion-promoting layer to the optically transparent layer made from oxides. Advantageously, organophosphoric acids and/or organophosphonic acids can interact with recognition elements, in particular with proteins or recognition elements coupled to proteins, and enhance binding of said recognition elements to the biochip.

Usable binding partners are preferably selected from the group comprising anti-mycotoxin antibodies, anti-mycotoxin-antibody conjugates, mycotoxins, mycotoxin conjugates, fragments of anti-mycotoxin antibodies, mycotoxin-binding peptides, mycotoxin-binding anticalins, mycotoxin-binding aptamers, mycotoxin-binding spiegelmers and/or mycotoxin-binding imprinted polymers, preferably selected from the group comprising anti-mycotoxin antibodies, anti-mycotoxin-antibody conjugates, mycotoxins and/or mycotoxin conjugates.

The binding partners interact in each case specifically with and/or with affinity to the in each case other binding partner. For example, anti-mycotoxin antibodies which are applied to a thin-layer waveguide bind with affinity to mycotoxins immobilized on said thin-film waveguide. Likewise, anti-mycotoxin antibodies immobilized on a thin-film waveguide bind with affinity to mycotoxins or mycotoxin conjugates which are applied to said thin-film waveguide. Binding specificity here depends on the affinity partners used. Thus, usable cross-reactive anti-mycotoxin antibodies bind with affinity to the corresponding mycotoxins, for example of the group of fumosins, but less specifically than, for example, a special antibody to fumosin B1 would. Binding partners which are immobilized are also referred to as recognition element or “capture molecules”.

Anti-mycotoxin-antibody conjugates and mycotoxin conjugates can be formed, for example, from a protein and anti-mycotoxin antibodies or mycotoxin.

In preferred embodiments, for example in indirectly competitive assays, the immobilized binding partners are mycotoxin conjugates. Mycotoxin conjugates may preferably be formed from mycotoxin bound to proteins, for example bovine serum albumin (BSA). A particular advantage of using such a mycotoxin-BSA conjugate is the fact that binding of the mycotoxin to the thin-film waveguide can be enhanced by an interaction between protein and organophosphoric acids and/or organophosphonic acids. This may improve adhesion of the recognition elements to said thin-film waveguide.

A labeling element, for example a fluorescent dye or fluorophore, may be bound directly to a binding partner, for example to an anti-mycotoxin antibody or a mycotoxin, or via a spacer element, for example a protein or an alkyl chain or polyethylene glycol chain. The labeling element, for example a fluorescent dye or fluorophore, is preferably bound to the mycotoxins via a protein. An example of a suitable protein is BSA. Binding of a fluorophore to a mycotoxin by means of BSA may distinctly improve binding of the labeling element to the binding partners, for example antibodies. Being able to avoid complicated processes for binding for example a fluorophore to a mycotoxin directly constitutes another advantage. Preferred binding partners for an immobilized anti-mycotoxin antibody, which may be used in a directly competitive assay, for example, are fluorescently labeled mycotoxin-BSA conjugates.

Mycotoxins may in principle be detected in samples, solutions or other media, all of which are capable of being applied to a thin-film waveguide. In preferred embodiments, the samples are human or animal food. Mycotoxins are preferably detected according to the process of the invention in cereals, cereal products, wine, juices or fruits and/or in products containing cereals, wine, juices and/or fruits. The sample to be analyzed, for example a food item or product, may here be applied to the thin-film waveguide or extracted with a solvent or solvent mixture, with the extracted extract being used. Said extract may be usable in diluted or concentrated form.

The mycotoxins may be removed from the sample to be studied, for example cereals or other food items, by treatment with a solvent or solvent mixture. For example, mycotoxins may be removed from grain samples by milling and subsequent extraction with water or organic solvents or solvent mixtures, for example with mixtures of water which may optionally be admixed with buffer substances, salts, acids or bases and other additives, and organic solvents, for example with mixtures of water and methanol or ethanol or water and acetonitrile. Other processes of extracting mycotoxins are known to the skilled worker. The dissolved mycotoxins obtained may then be analyzed either directly or after dilution or concentration on the thin-film waveguide or chip.

Usable recognition elements, also referred to as “capture molecules”, preferably selected from the group comprising anti-mycotoxin antibodies, anti-mycotoxin-antibody conjugates, mycotoxins and/or mycotoxin conjugates, preferably two or more different ones, may be immobilized covalently or noncovalently, for example by hydrophobic adsorption, on the thin-film waveguide surface or chip surface. They may be immobilized, for example, by applying the recognition elements by way of measurement fields, called spots, to the thin-film waveguide surface or chip surface, a process also referred to as spotting. Preference is given to spotting solution, preferably buffer solutions containing the binding partner(s) as recognition element, using devices for automatic application, called spotters. Preference is given to incubating the thin-film waveguides or chips after spotting for at least one hour, preferably some hours, so as to enable the recognition elements to attach to said thin-film waveguide or chip.

Preference is given to treating the biochips, after spotting, with a protein solution, preferably a solution of a usable blocking protein, for example BSA, for at least one hour, preferably 2 hours to 6 hours, particularly preferably 3 hours to 4 hours. After removing the solution, the thin-film waveguides or biochips may be dried and stored.

The sample and the preferably fluorescently labeled binding partner may be applied to the immobilized recognition elements on the thin-film waveguide, preferably evanescent field biochip, preferably a planar optical waveguide biochip, simultaneously or successively. Thus it is possible to add preferably fluorescently labeled binding partners, for example one or more preferably fluorescently labeled mycotoxins, mycotoxin conjugates or antibodies to one or more mycotoxins, prior to or during incubation of a sample, for example an extract, on the chip. It is also possible, for example, to apply to the chip an extract of a sample to be analyzed in a mixture with preferably labeled, preferentially fluorescently labeled, mycotoxins, mycotoxin conjugates or antibodies to one or more mycotoxins.

Particularly advantageously, the sample may be incubated according to the process of the invention with the immobilized binding partners as chemical or biochemical recognition element on the thin-film waveguide and/or the binding partners less than 15 minutes, preferably less than 10 minutes, particularly preferably less than 5 minutes, before detection of the signal.

This is greatly advantageous over known processes which require incubation times, with applied mycotoxin conjugate with a solution of labeled mycotoxin antibodies, of sometimes up to two hours or over processes which require the samples to be preincubated with labeled anti-mycotoxin antibodies. This enables the mycotoxins to be determined much more rapidly by the process of the invention than by known processes. More specifically, the incubation time can be shortened considerably. In particularly preferred embodiments, the incubation time may be less than 10 minutes or even only 5 minutes. This, in particular in combination with the further advantage of being able to dispense with washing steps, enables the process of the invention to produce a result in less than 20 minutes, preferably in less than 15, particularly preferably in less than 10, minutes.

Using the process of the invention, mycotoxins may be determined quantitatively and preferably with little variation. For example, the “interlaboratory coefficient of variation”, a measure of reproducibility, may be less than 50%, preferably less than 40%. Furthermore, the “intralaboratory coefficient of variation”, a measure of repeatability, may be less than 20%. This enables the process of the invention to be used within the framework of a standardized and simple process for determining mycotoxins in food items, for example cereals, cereal products or wine.

Detectable mycotoxins are preferably selected from the group comprising aflatoxins, ochratoxins, ergot alkaloids, patulin and/or fusarium toxins, for example selected from the group comprising deoxynivalenol, nivalenol, zearalenone, T-2 toxin, HT-2 toxin, ochratoxin A and/or fumonisins. Fumonisins are preferably selected from the group comprising fumonisin B1, fumonisin B2 and/or fumonisin B3.

Accordingly, usable binding partners are preferably selected from the group of mycotoxins comprising aflatoxins, ochratoxins, ergot alkaloids, patulin and/or fusarium toxins, for example selected from the group comprising deoxynivalenol, nivalenol, zearalenone, T-2 toxin, HT-2 toxin, ochratoxin A and/or fumonisins, and antibodies to mycotoxins selected from the group comprising aflatoxins, ochratoxins, ergot alkaloids, patulin and/or fusarium toxins, for example selected from the group comprising deoxynivalenol, nivalenol, zearalenone, T-2 toxin, HT-2 toxin and/or fumonisins.

Depending on the type of immunoassay used, one of the binding partners, for example one or more of the mycotoxins in the case of an indirectly competitive immunoassay, is immobilized as recognition element on the thin-film waveguide, while the other binding partner, for example one or more of the anti-mycotoxin antibodies in the case of an indirectly competitive immunoassay, is applied to the thin-film waveguide before or simultaneously with the sample. The binding partner to be added here is labeled preferentially luminescently, preferably with a fluorophore.

Usable binding partners are preferably selected from the group comprising deoxynivalenol, nivalenol, zearalenone, T-2 toxin, HT-2 toxin, ochratoxin A and/or fumonisin B1, fumonisin B2 and/or fumonisin B3, and antibodies to mycotoxins selected from the group comprising from the group comprising deoxynivalenol, nivalenol, zearalenone, T-2 toxin, HT-2 toxin, ochratoxin A and/or fumonisin B1, fumonisin B2 and/or fumonisin B3.

Preferably, monoclonal antibodies to mycotoxin, for example anti-fumosin B1, anti-fumosin B2 or anti-fumosin B3, may be used here. Antibodies acting against the group of fumosins may also be used. Usable binding partners, preferably antibodies to mycotoxins, may be used individually or in a mixture, and it is furthermore also possible to use cross-reactive antibodies.

A particular advantage of the process of the invention arises from the fact that the process of the invention can detect mycotoxins with increased sensitivity. For example, mycotoxins may be detectable even in the nanomolar or picomolar mycotoxin concentration range, in particular in human or animal food items, for example cereals, wine, juices, fruits and/or products therefrom, or in extracts of said food items or products. For example, mycotoxins may be detectable in cereal extract even in the range from 0.1 pM to 100 nM mycotoxin, preferably in the range from 1 pM to 1 nM mycotoxin. More specifically, concentrations of less than 1 nM, preferably less than 100 pM, mycotoxin, preferably less than 10 pM mycotoxin, particularly preferably less than 1 pM mycotoxin, may be detectable.

Furthermore, mycotoxins may be detectable in cereal extract in the range from 10⁻⁴ ppb to 10 000 ppb mycotoxin, in cereals in the range from 10⁻² ppb to 10 000 ppb mycotoxin. Preferentially, mycotoxins may be detectable in cereal extract in the range of ≦0.1 ppb mycotoxin, preferably in the range of ≦0.01 ppb mycotoxin, particularly preferably in the range of ≦10⁻⁴ ppb mycotoxin, in cereals in the range of ≦0.1 ppb mycotoxin, preferably in the range of ≦0.01 ppb mycotoxin, particularly preferably in the range of ≦10⁻⁴ ppb mycotoxin.

This enables the mycotoxins present in food items to be determined more accurately outside an analytical laboratory than previously possible.

The process of the invention enables at least two mycotoxins, preferentially from 2 to 1000 mycotoxins, preferably from 5 to 100 mycotoxins, to be detectable. More specifically, it is possible to determine mycotoxins simultaneously. This is a great advantage over known processes, most of which allow merely a single mycotoxin to be detected at a time.

A preferred embodiment of the process for detection of mycotoxins provides for immobilizing specific and/or affinity binding partners as chemical or biochemical recognition element for mycotoxins and/or a binding partner in a spatially separated manner on the surface of a thin-film waveguide comprising a first optically transparent wave-guiding layer (a) on top of a second optically transparent layer (b), with (b) having a lower refractive index than (a). The sample to be analyzed and the preferably fluorophore-labeled binding partners may then be added simultaneously or successively. The specific and/or affinity interaction between the binding partners immobilized on the thin-film waveguide, the mycotoxin(s) of the sample and/or the preferably fluorophore-labeled binding partners may be detected as a signal change in the evanescent field. The presence of a mycotoxin in the sample produces a change of the signal in the evanescent field.

According to the invention, the mycotoxins may be detected by an assay, for example an immunoassay, on the chip. Detection of the mycotoxins is preferentially carried out by way of an immunoassay, preferably a competitive immunoassay, for example a directly or indirectly competitive immunoassay, particularly preferably by way of an indirectly competitive immunoassay.

A preferred embodiment of the process for detection of mycotoxins by way of a directly competitive immunoassay may provide for immobilizing anti-mycotoxin antibodies as a chemical or biochemical recognition element for mycotoxins in a spatially separated manner on the surface of a thin-film waveguide comprising a first optically transparent wave-guiding layer (a) on top of a second optically transparent layer (b), with (b) having a lower refractive index than (a). Preferably fluorophore-labeled mycotoxins or preferably fluorophore-labeled mycotoxin-BSA conjugates may then be added simultaneously with or before the sample to be analyzed. The interaction between the anti-mycotoxin antibodies immobilized on the thin-film waveguide, the mycotoxin(s) of the sample and/or the preferably fluorophore-labeled mycotoxins or mycotoxin-BSA conjugates may be detected as a signal change in the evanescent field.

In the case of a direct competitive immunoassay, preferably two or more different anti-mycotoxin antibodies may be immobilized on the chip surface covalently or noncovalently, for example by spotting. Applying, for example, an extract of a sample to be studied in a mixture with preferably fluorescently labeled mycotoxins or mycotoxin conjugates to the chip results in said labeled or unlabeled mycotoxins or mycotoxin conjugates competing for the antibody binding sites available on said chip. The fluorescently labeled mycotoxins may be added prior to or during incubation of the extract on the chip. The amount of the labeled mycotoxins bound to the immobilized antibodies is inversely proportional to the amount of mycotoxins present in the extract.

Detection may also be conducted by way of a sandwich assay. In this case, labeled detection antibodies which bind to an immobilized complex of antibodies immobilized on the chip and mycotoxin are used rather than labeled mycotoxins or mycotoxin conjugates. In a sandwich assay, the amount of fluorophores bound to the antibodies is proportional to the concentration of mycotoxins in the extract.

Another preferred embodiment of the process for detection of mycotoxins by way of an indirectly competitive immunoassay may provide for immobilizing mycotoxins or preferably fluorophore-labeled mycotoxin-BSA conjugates as a chemical or biochemical recognition element in a spatially separated manner on the surface of a thin-film waveguide comprising a first optically transparent wave-guiding layer (a) on top of a second optically transparent layer (b), with (b) having a lower refractive index than (a). Preferably fluorophore-labeled anti-mycotoxin antibodies may then be added simultaneously with or before the sample to be analyzed. The interaction between the mycotoxins or preferably fluorophore-labeled mycotoxin-BSA conjugates immobilized on the thin-film waveguide, the mycotoxin(s) of the sample and/or the preferably fluorphore-labeled anti-mycotoxin antibodies may be detected as a signal change in the evanescent field.

According to the invention, mycotoxins may also be detected by an indirect, competitive immunoassay. In this case, mycotoxins or mycotoxin-conjugates, for example mycotoxin-protein conjugates, preferably mycotoxin-BSA conjugates, may be immobilized on the chip. Applying an extract of a sample to be studied in a mixture with preferably fluorescently labeled anti-mycotoxin-antibodies to the chip results in the immobilized mycotoxins and the mycotoxins in solution competing for the available binding sites of the fluorescently labeled antibodies. The fluorescently labeled anti-mycotoxin antibodies may be added prior to or during incubation of the extract on the chip. In this case, the amount of labeled antibodies bound is inversely proportional to the amount of mycotoxins present in the extract.

Furthermore, the signal can advantageously be detected in the evanescent field by means of a readout device. Said readout device may be, for example, a robust and inexpensive readout device.

Suitable software may be used for evaluating the signal intensity, for example fluorescence intensity, as well as calculating the amount of mycotoxins present in the sample.

The advantages provided by the process of the invention, in particular a combination of a process which is easy to carry out, the possibility being able to detect a plurality of mycotoxins simultaneously and quantitatively on a robust and inexpensive readout device, enable mycotoxins to be detected easily and rapidly outside an analytical laboratory.

The invention further relates to an apparatus for carrying out the process for detection of mycotoxins.

The apparatus for carrying out the process for detection of mycotoxins has a thin-film waveguide, preferably a planar optical waveguide biochip based on a thin-film waveguide comprising a first optically transparent wave-guiding layer (a) on top of a second optically transparent layer (b), with (b) having a lower refractive index than (a). The recognition elements are preferably immobilized on layer (a).

Examples of suitable planar optical waveguides are described in WO 01/92870 or in U.S. Pat. No. 5,959,292.

In preferred embodiments of the device, the optically transparent layer (b) of the thin-film waveguide, preferably planar optical waveguide biochip, may be made from silicates such as glass or quartz, or from a transparent plastic preferably selected from the group comprising polycarbonates, polyimides, polymethacrylates, polystyrenes, cyclic polyolefins and/or cyclic polyolefin copolymers, preferably from cyclic polyolefins or cyclic polyolefin copolymers. Examples of suitable plastics for preparing the optically transparent layer (b) are described in WO 03/020488.

Preference is given to transparent thermoplastic or injectable plastics, for example selected from the group comprising polycarbonate, polyimide, acrylate, in particular polymethyl methacrylate, or polystyrene.

In particular embodiments of the apparatus, the optically transparent wave-guiding layer (a) may comprise oxides selected from the group comprising TiO₂, ZnO, Nb₂O₅, Ta₂O₅, HfO₂ and/or ZrO₂, preferably selected from the group comprising TiO₂, Ta₂O₅ and/or Nb₂O₅. Combinations of several such oxides may also be used. Preference is given to an optically transparent wave-guiding layer (a) being made of TiO₂, ZnO, Nb₂O₅, Ta₂O₅, HfO₂ or ZrO₂, preferably TiO₂, Ta₂O₅ or Nb₂O₅. The use of tantalum pentoxide has proved particularly advantageous.

In preferred embodiments, the thin-film waveguide comprising, in particular on the optically transparent layer, oxides selected from the group comprising TiO₂, ZnO, Nb₂O₅, Ta₂O₅, HfO₂ and/or ZrO₂, comprises mono- or multilayers of organophosphoric acids of the following formula (I)

R—OPO₃H₂ (I)

and/or organophosphonic acids of the following formula (II)

R—PO₃H₂ (II)

and/or their salts, where

R is a C₁₀ to C₂₄ alkyl.

Preferably usable are organophosphoric acids and/or organophosphonic acids, preferably organophosphates and/or organophosphonates, where R is selected from the group comprising unbranched C₁₀ to C₂₀ alkyl, preferably selected from the group comprising unbranched C₁₂ to C₁₈ alkyl, preferably selected from the group comprising dodecylphsophoric acid, dodecylphosphate, octadecylphosphonate and/or octadecylphosphonic acid.

Preference is given to organophosphoric acids or organophosphates which can be applied by way of water-soluble salts from an aqueous solution to the thin-film waveguide.

In preferred embodiments, the organophosphoric acids and/or organophosphonic acids, preferably organophosphates, are applied by way of a monolayer to the thin-film waveguide, in particular an evanescent field biochip, preferably a planar optical waveguide biochip.

The monolayer may be applied as an adhesion-promoting layer to the optically transparent layer made from oxides. Advantageously, organophosphoric acids and/or organophosphonic acids can interact with recognition elements, in particular with recognition elements coupled to carrier proteins, and enhance binding of said recognition elements to the biochip.

In a preferred form of the apparatus, the organophosphoric acids and/or organophosphonic acids, preferably organophosphates, are applied to the thin-film waveguide, preferably to the optically transparent layer made of oxides, by way of an adhesion-promoting layer. Said adhesion-promoting layer may enhance binding of the recognition elements to the thin-film waveguide or biochip.

Preference is given to the adhesion-promoting layer having a thickness of less than 200 nm, preferably less than 20 nm.

Excitation light is preferably coupled into the optically transparent wave-guiding layer (a) by using one or more grating structures.

Said grating structure is preferably a relief grating with any profile, for example with a rectangular, triangular or semicircular profile, or a phase grating or volume grating with a periodic modulation of the refractive index in the essentially planar optically transparent layer (a). The grating structure may also be a diffractive grating with a uniform period or may be a multdiffractive grating. The grating structure may have a periodicity that varies in space perpendicularly or parallel to the direction of propagation of the excitation light coupled into the optically transparent wave-guiding layer (a).

Preference is given to the grating structures usable for incoupling of the excitation light having a period in the range from 200 nm to 1000 nm, preferably in the range from 200 nm to 400 nm. Furthermore, preference is given to the modulation transfer factor of the grating being in the range from 3 nm to 60 nm, preferably in the range from 10 nm to 40 nm. Preference is given to the ratio of modulation transfer factor to the thickness of the first optically transparent wave-guiding layer (a) being equal to or less than 0.4. Likewise, preference is given to refractive index modulation being pronouced both at the interface between layer a and layer b and at the interface of layer a to the analysis medium.

Preference is given to the optically transparent wave-guiding layer (a) having a thickness in the range from 40 nm to 1000 nm, preferably in the range from 40 nm to 300 nm, more preferably in the range from 80 nm to 200 nm.

The difference in refractive indices between layers (a) and (b) is preferentially ≧0.2, preferably ≧0.5, and more preferably 0.56.

The excitation light has a wavelength preferentially in the range from 300 nm to 1100 nm, preferably in the range from 300 nm to 800 nm, more preferably in the range from 500 nm to 700 nm.

Suitable excitation light may be coupled in via a grating structure, downstream of which, in the direction of propagation of the incoupled light guided in layer (a), there is a non-modulated region of layer (a), which contains an array of a multiplicity of measurement areas on which the various mycotoxins are detected. Downstream thereof, in the direction of propagation of the guided light, there may be advantageously one or more further grating structures with another array of measurement areas downstream thereof. Alternatively, the measurement areas of an array or else of a multiplicity of arrays may be in the modulated region of layer (a).

Preferably, to each downstream, in the direction of propagation of the incoupled excitation light, array of measurement areas there is assigned a grating structure for outcoupling said excitation light, which structure is specific for said array, it being possible for the grating structures to be formed specifically for individual arrays perpendicularly to the direction of propagation of the incoupled excitation light or else to extend across the entire thin-film waveguide in this direction.

The apparatus may have a very large number of individual measurement fields. In preferred embodiments of the apparatus, the specific and/or affinity binding partners as chemical or biochemical recognition element are applied by way of up to 100 000 measurement fields or spots in a two-dimensional arrangement, with a single measurement field or spot having an area preferably in the range from 0.001 mm² to 6 mm², preferentially in the range from 0.1 mm² to 1 mm². Preference is given to more than 10, preferably more than 50, measurement fields per square centimeter being applied to the thin-film waveguide or biochip.

The invention further relates to a kit for detection of mycotoxins. The kit comprises at least one thin-film waveguide comprising a first optically transparent wave-guiding layer (a) on top of a second optically transparent layer (b), with (b) having a lower refractive index than (a), to which waveguide specific and/or affinity binding partners are immobilized as a chemical or biochemical recognition element for mycotoxins and/or a binding partner in a spatially separated manner.

The kit may furthermore comprise at least one reagent comprising preferably labeled binding partners. The kit may also comprise a plurality of reagents comprising preferably labeled binding partners or a reagent comprising a mixture of different labeled binding partners. The kit may furthermore comprise buffers and/or solvents required for carrying out detection as claimed in any of the preceding claims. The invention may also provide for the kit to comprise a detection unit.

The kits may be used for rapid detection of mycotoxins.

An example which serves to illustrate the present invention is given below.

### EXAMPLE 1

Establishing a standard curve for measuring zearalenone in an indirectly competitive immunoassay on an evanescent field biochip

Seven biochips (Unaxis, Liechtenstein), with external dimensions of 1 cm×2 cm, made of glass into which an optical grating with a grating depth of 18 nm had been inscribed, and provided with a layer of 155 nm of tantalum pentoxide, were coated with octadecylphosphonic acid by dipping them into a solution of 500 μM of octadecylphosphonic acid in n-heptane/isopropanol (9:1). Conjugates of zearalenone and bovine serum albumin (ZEA-BSA, ZEA:BSA ratio=50:1, prepared by Biopure, Tulln, Austria) and BSA molecules labeled with the dye DyLight 647 (Pierce, Germany) (DyLight 647-BSA) were applied to the biochip with the aid of a spotter of the “Biochip Arrayer” (Perkin Elmer, Germany) type. The spotting solutions contained DyLight 647-BSA at a concentration of 5×10⁻⁴ mg/ml in PBS (137 mM NaCl, 2.8 mM KCl, 10 mM Na₂HPO₄, 1.8 mM KH₂PO₄, pH 7.4) containing 0.1% BSA and 0.1% Tween 20, 0.5 mg/ml BSA-ZEA conjugate in PBS containing 0.1% BSA and 0.1% Tween 20. The spots were applied to the chip in new alternating rows of in each case 10 DyLight 647-BSA spots and BSA-ZEA conjugate spots by way of two fields (arrays).

The spots were incubated at high humidity (40%) overnight and the biochips were then treated with a 3% strength solution of BSA in PBS for 4 hours. Measurement chambers were applied to the chips in such a way that two arrays with separated reaction chambers were formed on each chip. Aqueous solutions of zearalenone at various concentrations in the range from 0 μg/l to 31 μg/l were prepared and admixed with a monoclonal anti-zearalenone antibody (Biotez, Berlin), labeled with DyLight 647, thus producing in each case a 1 nM antibody solution.

The mixtures of different concentrations were in each case introduced into the measurement chambers, and the biochips were measured without further treatment steps on a “Minifluo IV” fluorescence reader (Bayer Technology Services, Germany) ten minutes or less. The fluorescence intensities obtained for each zearalenone spot were divided by the average of fluorescence intensities of the DyLight 647-BSA spots above and below the particular spot. The averages of the fluorescence intensities of all 40 spots of an array were determined. The concentration-dependent fluorescence intensities obtained were fitted by a sigmoidal fit wth the aid of the Origin 7G (Origin Lab Corporation, USA) computer program.

It was found that evaluation of the fluorescence intensity of the samples enabled the ZEA concentration to be quantified in a range from 0.4 ppb to 4 ppb zearalenone, corresponding to 80% and, respectively, 20% of maximum fluorescence intensity in the fitted sigmoidal curve, corresponding to a range from 1 nM to 10 nM of the ZEA concentration used in the solution.

### EXAMPLE 2

Establishing a standard curve for measuring deoxynivalenol (DON) and measuring a contaminated feed cereal sample

15 biochips (Unaxis, Liechtenstein) with external dimensions of 1 cm×2 cm, made of glass into which an optical grating (grating depth of 18 nm) had been inscribed, provided with a tantalum pentoxide layer (155 nm), were coated with octadecylphosphonic acid (by dipping them into a solution of octadecylphosphonic acid in n-heptane/isopropanol 9:1). Conjugates of deoxynivalenol and bovine serum albumin (DON-BSA, DON:BSA ratio=100:1, prepared by Biopure, Tulln, Austria) and dog IgG (Rockland, USA) were applied to the biochip with the aid of a spotter of the Nanoplotter (GeSiM, Germany) type. The spotting solutions consisted of dog IgG at a concentration of 0.2 mg/ml in PBS (137 mM NaCl, 2.8 mM KCl, 10 mM Na2HPO4, 1.8 mM KH2PO4, pH 7.4) containing trehalose, and of BSA-DON conjugate at a concentration of 1 mg/ml in PBS containing trehalose. The spots were applied to the chip in the form of two rows of in each case 12 dog IgG spots and a row of 12 BSA-DON conjugate spots in between by way of two fields (arrays).

The spots were incubated at 37° C. for 1 h, and the biochips were then treated with a solution of BSA in PBS for up to 4 hours. Measurement chambers were applied to the chips in such a way that two arrays with separate reaction chambers were formed on each chip. 5 g of non-contaminated wheat flour were extracted by shaking with a solution of 70% methanol in water (v/v) for 5 min. The extract was centrifuged and then diluted with a Tris citrate buffer (pH 7.4) containing BSA, caseine, low fat dry milk powder, Tween 20, polyethylene glycol and sucrose, in a ratio of 1:4 (v/v, extract:buffer). Solutions of deoxynivalenol at various concentrations (15 to 150 μg/l) were prepared and admixed with a monocloncal anti-deoxynivalenol antibody labeled with DyLight 647, and with a monoclonal goat anti-dog IgG antibody, likewise labeled with DyLight 647, thus producing in each case a 1 nM antibody solution.

The solutions of different concentrations were in each case introduced into the measurement chambers and the biochips were measured without further treatment steps on a “Minifluo IV” fluorescence reader (Bayer Technology Services, Germany) ten minutes of less. The fluorescence intensities obtained for each deoxynivalenol spot were divided by the average of fluorescence intensities of the dog IgG spot above and below the particular spot. The normalized averages of the fluorescence intensities of all 12 DON spots of an array were determined. The concentration-dependent, normalized fluorescence intensities obtained were fitted by a potential fit with the aid of a computer program.

Similarly to the extraction process illustrated above, 5 g of a DON-contaminated feed cereal meal sample (Coring, Germany, certified 526 ppb DON) were extracted, and the extract was diluted. To 300 μl of the diluted extract, a monoclonal anti-deoxynivalenol antibody labeled with DyLight 647 and a monoclonal goat anti-dog IgG antibody, likewise labeled with DyLight647, were added in such a way that in each case a 1 nM antibody solution was produced. In each case 100 μl of the solution were introduced into a measurement chamber, and the biochips were measured without further treatment steps on a “Minifluo IV” fluorescence reader (Bayer Technology Services, Germany) ten nimutes or less. The fluorescence intensities obtained for each deoxynivalenol spot were divided by the average of fluorescence intensities of the dog IgG spot above and below the particular spot. The normalized averages of the fluorescence intensities of all 12 DON spots of an array were determined. The fluorescence intensities obtained were converted with the aid of the above-described standard curve to DON concentrations in the feed cereal meal, resulting in an average of 590 ppb over the three measurements.

1. A process for rapid detection of mycotoxins, comprising the following steps:
a) providing a thin-film waveguide comprising a first optically transparent wave-guiding layer (a) on top of a second optically transparent layer (b), with (b) having a lower refractive index than (a), to which waveguide specific and/or affinity binding partners are immobilized as a chemical or biochemical recognition element for mycotoxins and/or a binding partner in a spatially separated manner,
b) applying a mycotoxin(s)-containing sample and binding partners to the immobilized binding partners on said thin-film waveguide,
c) detecting a signal in the evanescent field due to the interaction of the binding partners immobilized on the thin-film waveguide with the mycotoxins from the sample and/or with the binding partners, and
d) determining the amount of mycotoxin(s) present in the sample.

2. The process as claimed in claim 1, which further comprises applying to the thin-film waveguide mono- or multilayers of organophosphoric acids of the following formula (I)
R—OPO₃H₂ (I)
and/or organophosphonic acids of the following formula (II)
R—PO₃H₂ (II)
and/or their salts, where
R is a C₁₀ to C₂₄ alkyl.

3. The process as claimed in claim 2, wherein the organophosphoric acids, organophosphonic acids and/or their salts are selected from the group consisting of organophosphoric acids, organophosphonic acids, organophosphates and organophosphonates, with R being unbranched C₁₀ to C₂₀ alkyl.

4. The process as claimed in claim 1, wherein the thin-film waveguide comprises an optically transparent wave-guiding layer (a) comprising oxides selected from the group consisting of TiO₂, ZnO, Nb₂O₅, Ta₂O₅, HfO₂ and ZrO₂.

5. The process as claimed in claim 1, wherein the binding partners are selected from the group consisting of anti-mycotoxin antibodies, anti-mycotoxin-antibody conjugates, mycotoxins, mycotoxin conjugates, fragments of anti-mycotoxin antibodies, mycotoxin-binding peptides, mycotoxin-binding anticalins mycotoxin-binding aptamers, mycotoxin-binding spiegelmers and mycotoxin-binding imprinted polymers.

6. The process as claimed in claim 1, wherein a labeling element is bound to mycotoxins by means of a protein.

7. The process as claimed in claim 1, wherein the sample is a food item for humans or animals or an extract of said food items or products which has been extracted with a solvent or solvent mixture.

8. The process as claimed in claim 1, which further comprises incubating the sample with the immobilized binding partners as chemical or biochemical recognition element and/or the binding partners less than 15 minutes before detection of the signal.

9. The process as claimed in claim 1, wherein the mycotoxins are selected from the group consisting of aflatoxins, ochratoxins, ergot alkaloids, patulin and fusarium toxins.

10. The process as claimed in claim 1, which further comprises detecting the mycotoxins in cereal extract even in the range from 0.1 pM to 100 nM mycotoxin.

11. The process as claimed in claim 1, wherein the detection is carried out by way of an immunoassay.

12. An apparatus for carrying out a process for rapid detection of mycotoxins, the apparatus comprising a thin-film waveguide comprising a first optical transparent wave-guiding layer (a) on top of a second optical transparent layer (b), with (b) having a lower refractive index than (a).

13. The apparatus as claimed in claim 12, wherein the optically transparent layer (b) of the thin-film waveguide comprising a first optically transparent wave-guiding layer (a) on top of a second optically transparent layer (b), with (b) having a lower refractive index than (a), is made from silicates or from a transparent plastic.

14. The apparatus as claimed in claim 12, wherein the optically transparent wave-guiding layer (a) has a thickness in the range from 40 nm to 1000 nm.

15. The apparatus as claimed in claim 12, wherein excitation light is coupled into the optically transparent wave-guiding layer (a) by using one or more grating structures.

16. The apparatus as claimed in claim 15, wherein the grating structures usable for coupling in excitation light have a period in the range from 200 nm to 1000 nm.

17. The apparatus as claimed in claim 15, wherein the grating has a modulation transfer factor in the range from 3 nm to 60 nm.

18. The apparatus as claimed in claim 15, wherein the excitation light has a wavelength in the range from 300 nm to 1100 nm.

19. The apparatus as claimed in claim 12, which further comprises applied to the thin-film waveguide mono- or multilayers of organophosphoric acids of the following formula (I)
R—OPO₃H₂ (I)
and/or organophosphonic acids of the following formula (II)
R—PO₃H₂ (II)
and/or their salts, where
R is a C₁₀ to C₂₄ alkyl.

20. The apparatus as claimed in claim 12, wherein recognition elements are applied to the thin-film waveguide by way of up to 100 000 measurement fields in a two-dimensional arrangement.

21. The apparatus as claimed in claim 20, wherein more than 10 measurement fields per square centimeter are applied to the thin-film waveguide.

22. A kit for rapid detection of mycotoxins, wherein the kit comprises at least one thin-film waveguide comprising a first optically transparent wave-guiding layer (a) on top of a second optically transparent layer (b), with (b) having a lower refractive index than (a), to which waveguide specific and/or affinity binding partners are immobilized as a chemical or biochemical recognition element for mycotoxins and/or a binding partner in a spatially separated manner.

23. The kit as claimed in claim 22, which comprises a.

24. (canceled)
